(19) United States
(12) Patent Application Publication
(10) Pub. No.: US 2022/0229003 A1
(43) Pub. Date: Jul. 21, 2022

(54) A METHOD INVOLVING MEASURING OF WATER QUALITY AND/OR DETECTION OF ONE OR MORE SUBSTANCES IN A WATER FLOW

(71) Applicant: ORBITAL SYSTEMS AB, Malmo (SE)

(72) Inventors: Andreas ANDERBERG, Staffanstorp (SE); Mikael NILSSON, Trelleborg (SE)

(21) Appl. No.: 17/609,508

(22) PCT Filed: May 6, 2020
(86) PCT No.: PCT/SE2020/050467

§ 371 Date: Nov. 8, 2021

(30) Foreign Application Priority Data
May 8, 2019 (SE) 1950548-6

(51) Int. Cl. G01N 27/06; G01N 33/18

(52) U.S. Cl. CPC G01N 27/06; G01N 33/18

(57) ABSTRACT

The present invention describes a method involving measuring of water quality and/or detection of one or more substances in a water flow, said method involving the steps of using a sensor system comprising at least two electrodes, for sending frequency from at least one electrode and receiving a response from at least another electrode, wherein the method involves filtration over one or more frequency ranges in the response, to measure the impedance and using the impedance as an indicator of the water quality and/or for detection of one or more substances in the water flow.

### FIELD OF THE INVENTION

The present invention relates to a method involving measuring of water quality and/or detection of one or more substances in a water flow.

### TECHNICAL BACKGROUND

It has long been known to measure water quality in water flows. One example is disclosed in WO18097789, which discloses a sensor system intended for a system allowing for purification and recycling of water or separation of water, wherein said system allowing for purification and recycling of water or separation of water comprises a water treatment unit, wherein said sensor system comprises one first sensor type directed to indicating the function of a water treating source in the water treatment unit, and wherein the sensor system also comprises a second sensor type directed to indicating the water quality, and wherein both the first sensor type and the second sensor type give input to a control system of the system with respect to a selection decision of either recycling of water in the system or separation of water from the system.

The present invention is also directed to measuring water quality in a water flow, e.g. in a water recirculation system, such as in a shower. Therefore, one aim of the present invention is to provide an effective method for measuring water quality in a water flow, such as in applications where water is flowing, e.g. in water recirculation systems.

### SUMMARY OF THE INVENTION

The stated purpose above is achieved by a method involving measuring of water quality and/or detection of one or more substances in a water flow, said method involving the steps of using a sensor system comprising at least two electrodes, for sending frequency from at least one electrode and receiving a response from at least another electrode, wherein the method involves filtration over one or more frequency ranges in the response, to measure the impedance and using the impedance as an indicator of the water quality and/or for detection of one or more substances in the water flow.

According to the present invention, the electrodes suitable are implemented as one electrode pair. Also several electrodes and several electrodes are possible, as will be clear from the description below.

It should be noted that in the method according to the present invention, the change of conductivity is measured in the form of impedance. This measure is used as an indicator of the water quality and/or as an indicator of a substance.

Using impedance in general has been performed before in the technology relating to water quality. For instance in U.S. Pat. No. 4,853,638 there is disclosed an apparatus and method for measuring an electrical conductivity of an aqueous solution by applying an AC voltage between measuring electrodes which are dipped in said aqueous solution containing ions and measuring at least one of an electrode surface reaction resistance and a liquid resistance of said aqueous solution. The apparatus comprises a plurality of electrode pairs having respective electrode intervals different from each other and having substantially identical electrochemical, electrode surface reaction resistances. Furthermore, in U.S. Pat. No. 4,853,638 there is also disclosed a method of measuring an electrical conductivity of an aqueous solution comprising the steps of immersing at least a pair of electrodes in said aqueous solution under measurement in order to obtain a relationship between an electrical conductivity and a temperature by measuring electrical conductivities at least at two different temperatures To and Tn with respect to said aqueous solution under measurement, and measuring a complex AC impedance between said pair of electrodes at each of the temperatures of said aqueous solution by applying an AC voltage between said pair of electrodes while varying a frequency of the AC voltage, wherein the measurement temperature To is in a first range, and the measurement temperature Tn is in a second range different from said first range, and at least one value of the electrical conductivity is measured in each of the temperature ranges; and inter alia obtaining a liquid resistance of said aqueous solution under measurement at each of the measurement temperatures from a frequency response of each of the complex impedance.

One main difference between the method according to the present invention and the device and method according to U.S. Pat. No. 4,853,638 relates to that the method according to the present invention involves filtration over one or more frequency ranges in the response. This is not disclosed or hinted in U.S. Pat. No. 4,853,638, and filtration is a key and essential feature of the present invention as will become evident from the description below.

According to the present invention, change in conductivity may be used as a measure for water quality. This change of conductivity may be regarded as a water quality parameter to use according to the present invention. Furthermore, the method according to the present invention may also be used as a starting point to enable to identify one or more substances present in the water flow.

### SPECIFIC EMBODIMENTS OF THE INVENTION

Some specific embodiments of the present invention are disclosed below.

According to one specific embodiment of the present invention, said at least two electrodes are positioned at a distance opposite or substantially opposite each other and a first electrode sends a frequency and a second electrode receives a response. As hinted above, one electrode pair may be arranged with two electrodes opposite each other.

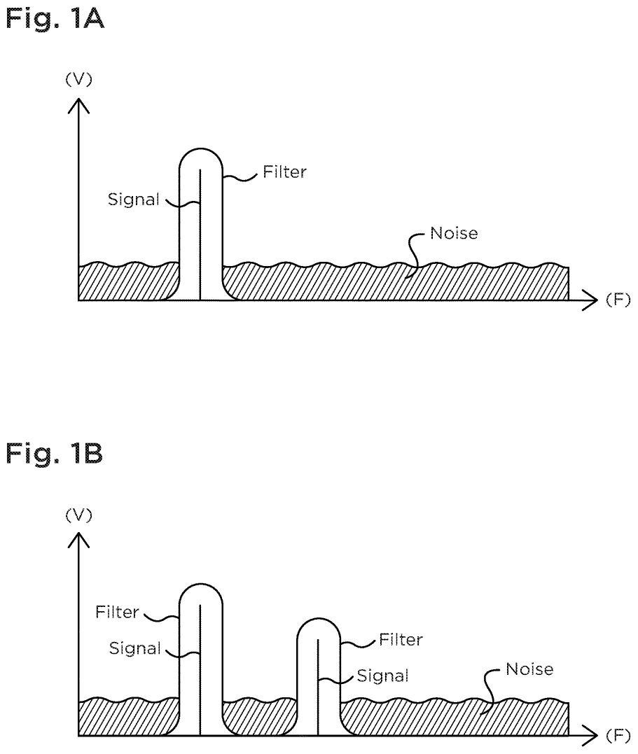

As mentioned above, the method according to the present invention involves filtration over one or more frequency ranges in the response. As notable in FIG. 1 this may be performed over one or several frequencies. Moreover, according to the present invention the method may involve analyzing frequencies close to and/or inside the one or more frequency ranges which are filtrated over.

According to yet another embodiment, sending a frequency involves sending a sinusoidal frequency signal. Moreover, according to one specific embodiment of the present invention, sending a frequency involves sending a sinusoidal frequency signal and where the method involves filtration over one or more filtration ranges in the response.

Furthermore, the method according to the present invention may also involve sending multiple sinusoidal signals in different frequencies and where the method also involves matching multiple filtrations in the response. In such a case the sinusoidal signals used are each matched with suitable frequency filters in the response.

Besides certain frequency ranges, also a so called sweep may be used. In line with this, according to one specific embodiment of the present invention, the method involves sending at least one frequency sweep between two frequencies and receiving a response over said at least one frequency sweep. In relation to using a frequency sweep in the method according to the present invention it should be noted that it is important to ensure that the signal received, that is also after being filtered, corresponds to the right signal sent. This is of course important and valid also for other embodiments according to the present inventions when several frequency signals are being sent and received.

Also other alternatives are possible. According to one specific embodiment of the present invention, the method involves sending a frequency noise between two frequencies and receiving a response over said at least one frequency noise. Both in the case of using a sweep or a noise, the actual the filtration may be performed over one or more filtration ranges in the response. In the case of using a noise, different kinds may be used. A so called randomized white noise is one alternative.

The filtration(s) according to the present invention may be performed by use of different technologies. According to one specific embodiment of the present invention, wherein the filtration involves applying a FT (Fourier Transform), FIR (Finite Impulse Response), IIR (Infinite Impulse Response), or a combination thereof. As hinted, combinations of the different alternatives are totally possible. Different alternatives are better for certain signal technologies. For instance, the use of Fourier Transforms is very suitable on signals with broad bands, e.g. a sweep.

Also different frequency levels may be used according to the present invention. According to one specific embodiment, frequencies used are in the range of from 0-100 kHz.

Furthermore, according to yet another specific embodiment of the present invention, the method involves sending different frequencies and receiving responses for each frequency used. This further indicates the matchmaking between a certain signal and the response therefore. Moreover, filters may be used for each signal sent and response received.

Moreover, according to yet another embodiment, more than two electrodes are used and wherein at least two electrodes send frequencies at different levels. According to one specific embodiment, each electrode send a frequency or frequency range not used for another electrode sending.

To combine different frequency ranges or frequency sending technologies may be of interest according to the present invention. According to one specific embodiment of the present invention, the method involves at least two individual method steps which include sending frequencies, preferably as one or more sinusoidal signals, frequency noises or frequency sweeps or a combination thereof, and receiving multiple responses. This is one embodiment in which combinations of different frequency sending technologies are combined. The filters used should be matched accordingly.

The method according to the present invention may be used in different types of applications. Water recirculation systems are one suitable application. In line with this, according to one specific embodiment, the method is performed in a water recirculation system intended for recycling of water or discarding of water not suitable to recycle, said water recirculation system comprising a flow path for recirculation, at least one water treating unit, and a sensor unit arranged for measurement of at least water quality, and wherein the sensor unit is connected to the control unit which decides if water should be recycled or discarded in a point of separation based on the measurement of water quality, said water recirculation system also comprising a heating source and a user outflow arranged at the end of the flow path for recirculation. This is further explained below in relation to the embodiment shown in the drawings.

### DESCRIPTION OF THE DRAWINGS

In FIG. 1A there is shown a graph representing a first generic alternative according to one embodiment of the present invention. On the y axis the voltage (V) is shown, and one the x axis the frequency (F) is shown. It should be noted that the voltage is a direct measure of the impedance according to standard equations in laws of science of electricity and physics.

As you will see, in this case the method involves sending on frequency (“signal”) from one electrode which is then received by another electrode. In the method, a filter is used. This filter filtrates over only a small range of the frequencies, where the signal is within this range. Moreover, the noise is also depicted in the graph. As is evident, the relationship of signal to noise will be better when using the method according to the present invention.

As an example, the actual signal may be in the form of sinusoidal frequency signal, as is described above.

In FIG. 1B there is shown another embodiment according to the present invention. This embodiment is corresponding to the one shown in FIG. 1A, however in this case there are two different signals/frequencies sent and received and also two corresponding filters used.

In FIG. 1C there is shown another embodiment of the method according to the present invention. In this case, the graph is intended to show a method wherein a frequency sweep is used. As notable, then different signals are sent in a specific frequency range. Moreover, for all new signals sent, then a corresponding filter with a frequency range corresponding to the sent signal is used for filtration.

As may be understood from above, when using several signals and filters, the method according to the present invention may be improved. This alternative according to the present invention implies that more valuable data may be obtained during a reasonable time. If the method instead would involve using the entire frequency band range, then such a sweep and the data handling thereafter would take a comparatively long time. For instance, when operating a water recirculation system based on water quality measurements by use of a method according to the present invention, then it is of interest to ensure a short response time. By using several frequency ranges or frequencies of great data interest and excluding others of no data interest, then the response time may be shortened. Again, the method according to the present invention still provides very valuable data in certain set frequency ranges suitable when deciding water quality in a water flow, and does so in a very fast response time.

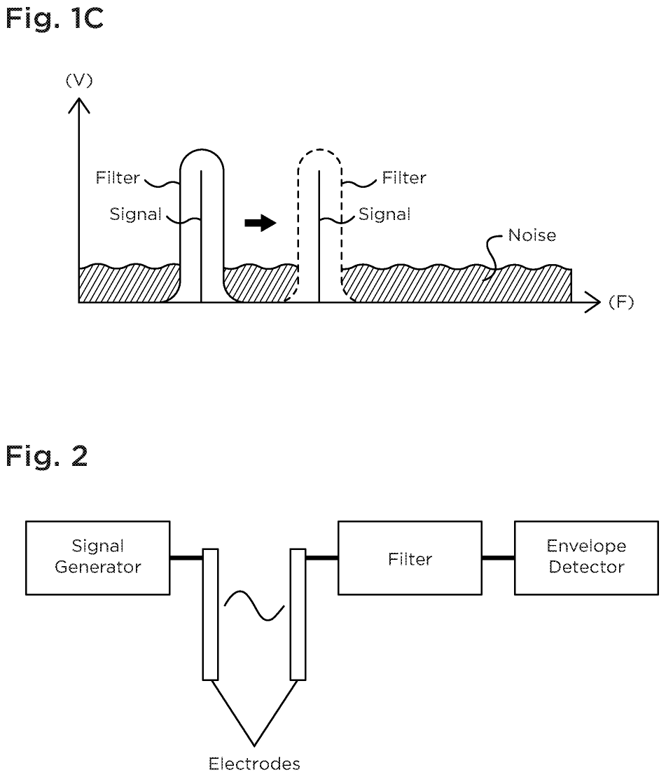

In FIG. 2 there is shown a possible set-up according to one embodiment according to the present invention. A signal generator enables a signal to be generated in one electrode. Another electrode, arranged opposite the electrode providing the signal, receives the signal. As seen, a filter is used to ensure to filtrate a frequency range suitable to match the signal sent. Moreover, an envelope detector is used to transform the received and filtrated signal to a new type of signal which is based on the amplitude in the received and filtrated signal. Therefore, according to one specific embodiment of the present invention, an envelope detector transforms a received and filtrated signal to a new signal based on the amplitude of the received and filtrated signal. Furthermore, according to yet another specific embodiment of the present invention, the signal from the envelope detector is used to identify one or more amplitude peaks and/or changes in the amplitude of the received and filtrated signal. To obtain such data based on the amplitude may be valuable when measuring water quality, e.g. in a water flow in water recirculation system such as further mentioned below.

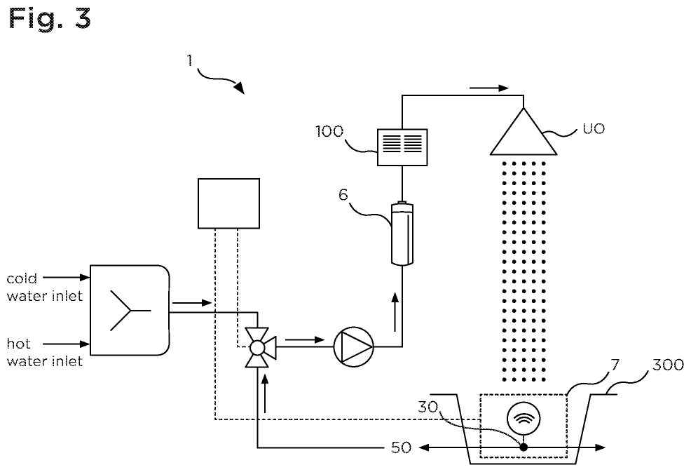

In FIG. 3 there is shown a water recirculation system in which the method according to the present invention suitably may be used. According to one embodiment, the water recirculation system 1, intended for recycling of water or discarding of water not suitable to recycle, comprises a flow path for recirculation 50, at least one water treating unit 6, and a sensor unit 7 arranged for measurement of at least water quality, where the sensor unit 7 is connected to the control unit which decides if water should be recycled or discarded in a point of separation 30 based on the measurement of water quality, said water recirculation system 1 also comprising a heating source 100 and a user outflow UO arranged at the end of the flow path for recirculation 50. As may be noted, in this case the water recirculation system is in the form of a shower, however also other applications are possible, e.g. sinks or integrated systems with several such components.

1. A method involving measuring of water quality and/or detection of one or more substances in a water flow, said method involving the steps of using a sensor system comprising at least two electrodes, for sending frequency from at least one electrode and receiving a response from at least another electrode, wherein the method involves filtration over one or more frequency ranges in the response, to measure the impedance and using the impedance as an indicator of the water quality and/or for detection of one or more substances in the water flow.

2. The method according to claim 1, wherein said at least two electrodes are positioned at a distance opposite or substantially opposite each other and a first electrode sends a frequency and a second electrode receives a response.

3. The method according to claim 1, wherein sending a frequency involves sending a sinusoidal frequency signal.

4. The method according to claim 1 wherein the method involves sending multiple sinusoidal signals in different frequencies and wherein the method involves matching multiple filtrations in the response.

5. The method according to claim 1, wherein the method involves sending at least one frequency sweep between two frequencies and receiving a response over said at least one frequency sweep.

6. The method according to claim 1, wherein the method involves sending a frequency noise between two frequencies and receiving a response over said at least one frequency noise.

7. The method according to claim 5, wherein the filtration is performed over one or more filtration ranges in the response.

8. The method according to claim 1, wherein the filtration involves applying a FT (Fourier Transform), FIR (Finite Impulse Response), IIR (Infinite Impulse Response), or a combination thereof.

9. The method according to claim 1, wherein frequencies used are in the range of from 0-100 kHz.

10. The method according to claim 1, wherein the method involves sending different frequencies and receiving responses for each frequency used.

11. The method according to claim 1, wherein more than two electrodes are used and wherein at least two electrodes send frequencies at different levels.

12. The method according to claim 1, wherein the method involves at least two individual method steps which include sending frequencies, preferably as one or more sinusoidal signals, frequency noises or frequency sweeps or a combination thereof, and receiving multiple responses.

13. The method according to claim 1, wherein an envelope detector transforms a received and filtrated signal to a new signal based on the amplitude of the received and filtrated signal.

14. The method according to claim 13, wherein the signal from the envelope detector is used to identify one or more amplitude peaks and/or changes in the amplitude of the received and filtrated signal.

15. The method according to claim 1, wherein the method is performed in a water recirculation system intended for recycling of water or discarding of water not suitable to recycle, said water recirculation system comprising a flow path for recirculation, at least one water treating unit, and a sensor unit arranged for measurement of at least water quality, and wherein the sensor unit is connected to the control unit which decides if water should be recycled or discarded in a point of separation based on the measurement of water quality, said water recirculation system also comprising a heating source and a user outflow arranged at the end of the flow path for recirculation.
